(19) United States
(12) Patent Application Publication
(10) Pub. No.: US 2010/0239707 A1
(43) Pub. Date: Sep. 23, 2010

(54) Increased Meat Tenderness Via Induced Post-Mortem Muscle Tissue Breakdown

(76) Inventors: David Goldberg, Boulder, CO (US); Keith E. Belk, Fort Collins, CO (US); Phillip D. Bass, Sterling, OH (US); Hyungchul Han, Fort Collins, CO (US); Joseph D. Tatum, Windsor, CO (US); Gary L. Mason, Fort Collins, CO (US)

Correspondence Address: LATHROP & GAGE LLP, 4845 PEARL EAST CIRCLE, SUITE 201, BOULDER, CO, 80301, US

(21) Appl. No.: 12/682,766

(22) PCT Filed: Oct. 9, 2008
(86) PCT No.: PCT/US08/11608

§ 371 Date: Jun. 3, 2010

Related U.S. Application Data
(60) Provisional application No. 61/005,507, filed on Dec. 5, 2007.
(60) Provisional application No. 61/005,605, filed on Dec. 6, 2007.
(60) Provisional application No. 61/005,145, filed on Dec. 3, 2007.
(60) Provisional application No. 60/998,295, filed on Oct. 10, 2007.

(51) Int. Cl. A23K 1/18; C07C 57/00; C07C 53/00; C07C 215/24; C07C 233/19; A23L 1/31; C01B 15/01; C01B 13/00; B01J 23/745; C09K 3/00

(52) U.S. Cl. 426/2; 562/599; 562/606; 564/507; 564/224; 426/652; 423/584; 423/581; 502/338; 252/182.12

(57) ABSTRACT

The present methods and compounds relate to increasing meat tenderness by inducing post-mortem breakdown in muscle tissue. This is achieved by the use of beta-blockers in the pre-mortem period, which results in higher calpastatin levels, and reduced hyperplasia. This is also achieved by the use of agents immediately before slaughter that induce apoptosis in muscle tissue. This is further achieved by administering agents that induce muscle fiber fission.

### CROSS REFERENCE TO RELATED PATENT APPLICATIONS

This application is related to and claims priority from Provisional Patent Application No. 60/998,295, filed Oct. 10, 2007, and titled “Increased Meat Tenderness via Induced Apoptosis”, from Provisional Patent Application No. 61/005,145, filed Dec. 3, 2008, and titled “Increased Meat Tenderness via Induced Apoptosis”, from Provisional Patent Application No. 61/005,507, filed Dec. 5, 2008, and titled “Increased Meat Tenderness via Induced Muscle Fission”, and from Provisional Patent Application No. 61/005,605, filed Dec. 6, 2008, and titled “Use of Beta-Blockers to Increase Meat Tenderness”.

### TECHNICAL FIELD

The present invention relates to increasing meat tenderness by inducing post-mortem breakdown in muscle tissue.

### BACKGROUND

The single most important quality affecting consumer satisfaction of meat is its tenderness. Experimental economic studies indicate that consumers would be willing to pay an additional 10-20% for guaranteed tender ribeye or top-loin steaks. The total potential increase in carcass value has been estimated by these studies at up to 15-20% per carcass.

This preference for tender beef has a number of important consequences. For example, Bos indicus cattle tend to be less tender than that of Bos taurus cattle, leading to generally lower prices for Bos indicus cattle and particularly for those that contain over ⅜ Bos indicus bloodlines. Also, since grain finishing tends to produce more tender meat, corn-finishing is common, leading to higher grain and meat prices.

Meat toughness is anticipated to increase in the next years. For example, increased grain prices due to competition with corn-based ethanol production is leading to decreased grain finishing. Furthermore, beta-agonists are increasingly being used to increase meat production through improvements in feed efficiency. Their use, however, is often accompanied by an increase in toughness. Industry groups state that meat toughness is the leading non-safety issue.

There has been considerable effort over a period of decades to find pre-harvest or post-harvest methods for managing meat tenderness. The more important improvements have come from post-mortem electrical stimulation to reduce toughness, but this has only limited effectiveness. A method that reliably and safely increased meat tenderness would have profound impact on the industry by improving quality and customer satisfaction, which could increase meat sales and improve the market price for meat products. In addition, such a method could have an important impact on the cost of meat production. As mentioned above, reduced corn feed finishing and the use beta-agonists (which result in higher feed efficiency) both reduce input costs, but at the same time, result in tougher meat. However, if there were a means of making meat prepared with these pre-harvest management techniques more tender, producers could make use of these cost saving methods and still have acceptably tender meat.

Finally, it should be noted that less corn in finishing, the use of beta-agonists, and the specific breeds can result in leaner meat that is potentially healthier. However, the current grading methodology encourages consumers to avoid such meat because of the perceived tenderness of highly marbled meat. Having a means of effectively and safely tenderizing meat would, by reducing the linkage between tenderness and marbling (as marbling is a prime determinant of flavor), result in a new range of choices for consumers who are willing to balance flavor and healthfulness without sacrificing tenderness.

Therefore, it would be of great benefit to the meat industry and to consumers if there was a safe means for tenderizing meat that could be applied generally to animals or carcasses. It is to this goal that the current methods are directed.

### SUMMARY OF THE INVENTION

It would be preferable for the present invention to provide a method for increasing meat tenderness.

It would also be preferable for the present invention to provide a method for increasing meat tenderness that can be implemented in production environment.

It would further be preferable for the present invention to provide a method for increasing meat tenderness that is economically profitable.

It would additionally be preferable for the present invention to provide a method for increasing meat tenderness that does not hurt the safety of consuming the meat.

Additional objects, advantages and novel features of this invention shall be set forth in part in the description that follows, and will become apparent to those skilled in the art upon examination of the following specification or may be learned through the practice of the invention. The objects and advantages of the invention may be realized and attained by means of the instrumentalities, combinations, and methods particularly pointed out in the appended claims.

In accordance with the purposes of the present invention, as embodied and broadly described herein, the present invention is generally directed to a method for inducing tenderness in meat derived from an animal by treatment of the animal prior to its death, which comprises administering to the animal a beta-blocker, providing a predetermined time without beta-blocker administration to allow for clearing of the beta-blocker from the animal, and slaughtering the animal.

The predetermined time can be less than 4 days. The predetermined period of time can allow at least 90% of the beta-blocker administered to be cleared from the animal.

The dosage of beta-blocker administered can be greater than 50% of the normal human dosage of the equivalent beta-blocker.

The administering can be performed via oral administration, implantation, placement as a suppository, intravenous injection or intramuscular injection. In addition, beta-agonist can be provided to the animals, wherein the provision of beta-agonist is prior to the administration of the beta-blocker.

The present invention is also directed to a method for inducing tenderness in meat derived from an animal by treatment of the animal prior to its death, comprising administering to the animal an apoptosis inducer and slaughtering the animal.

The apoptosis inducer can comprise an extrinsic inducer or an immune inducer. It can also comprise an oxidizing agent, and the oxidizing agent can be hydrogen peroxide and ozone, and can further comprise a peroxide metabolase inhibitor.

The apoptosis inducer can comprise a lipid, which can comprise a free fatty acid, ceramide, or sphingosine. The lipid can be solubilized in aqueous solution with an emulsifier. Also, the lipid can be administered as a solution in a solvent that can comprise ethanol, n-butanol, or iso-butanol.

The apoptosis inducer can comprises a peroxide and a ferrous or ferric salt. In addition, the apoptosis inducer can comprise a peroxide and a lipid, which can also comprise a ferrous or ferric salt.

In addition, beta-agonist can be provided to the animals, wherein the provision of beta-agonist is prior to the administration of the beta-blocker.

The present invention can additionally be directed to a method for inducing tenderness in meat derived from an animal by treatment of the animal prior to its death, comprising administering to the animal a muscle fiber fission inducer and slaughtering the animal.

The muscle fiber fission inducer can be selected from the molecular families of myoseverin and nocodazole. The muscle fiber fission inducer can have a ratio of oral to intravenous LD50 of more than 100. The muscle fiber fission inducer can have an in vivo activity half-life of less than 12 hours.

Additionally, beta-agonist can be provided to the animals, wherein the provision of beta-agonist is prior to the administration of the beta-blocker.

The present invention can yet also be directed to a compound for administration to a live animal for increasing post-mortem meat tenderness, comprising an apoptosis inducer.

The apoptosis inducer can comprise an oxidizing agent, which can further comprise a peroxide metabolase inhibitor. The oxidizing agent can be selected from the group consisting of hydrogen peroxide, ozone, and Fenton's reagent.

The apoptosis inducer can comprise a lipid, which can be selected from the group consisting of free fatty acid, ceramide and sphingosine.

The apoptosis inducer can comprise an oxidizing agent and a lipid.

The present invention can yet additionally be directed to a compound for administration to a live animal for increasing post-mortem meat tenderness, comprising an oxidizing agent.

The present invention can yet further be directed to a compound for administration to a live animal for increasing post-mortem meat tenderness, comprising an oxidizing agent.

The present invention can yet also be directed to a compound for administration to a live animal for increasing post-mortem meat tenderness, comprising a beta-blocker.

The present invention can yet further be directed to a compound for administration to a live animal for increasing post-mortem meat tenderness, comprising a muscle fiber fission inducer. The muscle fiber fission inducer can inhibit microtubule polymerization. The muscle fiber fission inducer can also have a ratio of intravenous to oral LD₅₀ greater than 25.

The muscle fiber fission inducer can comprise a hydrolysable component with an in vivo half-life of less than a predetermined time. The predetermined time can be less than 12 hours.

The muscle fiber fission inducer can also comprise a highly reactive substituent with an in vivo half-life of less than a predetermined time. The predetermined time can be less than 12 hours.

### BEST MODE FOR CARRYING OUT THE INVENTION

### Overview

The methods of the present invention relate to increasing muscle tenderness via induced muscle tissue breakdown. In normal post-mortem muscle, the cells die and breakdown. The methods of the present invention either enhance this normal breakdown, introduce a new process by which breakdown occurs, or change the differentiation state of the muscle tissue so as to favor increased meat tenderness.

### Enhancing Normal Muscle Tissue Breakdown through Beta-Blockers

Let us start by considering the sometimes conflicting goals of a feedlot operator. The operator wishes to increase the conversion of feed into lean muscle mass (the feed efficiency). At the same time, the operator wishes to increase the tenderness of the meat to increase its palatability and value to the end-customer. In practice, as we will see, increasing the feed efficiency is often at odds with increasing tenderness.

The first goal of improving the feed efficiency can be accomplished through two means. In the first case, the diameter and volume of the individual muscle fibers can be increased, in a process called hypertrophy. The downside of hypertrophy is that it is known that increasing muscle fiber diameter decreases meat tenderness.

A preferable means of increasing muscle mass would be to engage in hyperplasia, which is an increase in the number of muscle fibers. This would result in greater muscle mass, but would not decrease (and indeed, might increase) meat tenderness.

As mentioned above, the use of beta-adrenergic agonists (hereinafter “beta-agonist”) has the effect of increasing muscle mass at the cost of increasing meat tenderness. The mechanism by which the beta-agonists exert this effect is not completely understood, but the effects appear to be related to beta-agonists antagonistic effects on myostatin resulting in muscle hypertrophy, as well as increasing the activity of calpastatin, which reduces post-mortem proteolysis.

The present invention teaches the use of beta-adrenergic antagonists (beta-blockers) as a means of increasing meat tenderness. This will have a number of positive direct physiological effects. In a first effect, beta-blockers will reduce the hypertrophic effects. Since weight gain will still be significant over the course of feedlot treatment, we expect that the ratio of hyperplasia to hypertrophy will be greater than that in beta-agonist treated animals.

In a second effect, calpastatin activity will be reduced in beta-blocker treated cows, inversely to the way that it is increased in beta-agonist treated cows. This will have the effect of increasing post-mortem proteolysis, and thereby increase meat tenderness.

In a third effect, the degree of marbling will be increased, as has been empirically found in many studies. While the degree of marbling has only a modest effect on tenderness, it has a substantial effect in terms of the taste and overall palatability of meat, and it is highly desired by a substantial fraction of consumers.

In a fourth effect, beta-antagonists are known to have adverse health effects, with particular note to be made regarding immune suppression. The studies to date on the use of beta-agonists for feed efficiency have been controlled studies, in which animals are in conditions far less crowded and stressed than feedlots, and therefore where animal health is of lower concern. It should be noted that beta-agonists not only suppress the immune system directly, but also by increasing the overall aggression of animals, makes for higher behavioral stress, resulting in a higher level of stress hormones. These stress hormones also lead to lower immune functioning and poorer disease response. Animals will exhibit more illnesses (e.g. respiratory, hoof/mobility, general viral, bacterial, fungal or mycoplamal infections), and will respond slower and less aggressively to medical treatment. On the other hand, the use of beta-blockers will tend to improve immune function and lower stress hormones to the health benefit of the animals.

Since most studies on the effects of beta-agonists on muscle mass have been performed on research animals in controlled environments, and given that sick and stressed animals gain muscle slowly (and may lose muscle mass), it may be that in feedyard environments where animal health an important aspect of performance, the increased illness that is expected from the use of beta-agonists may result in lower overall improvement in meat production due to beta-agonists in actual commercial use than has been shown in controlled studies.

### Beta-Blocker and Dosage

The beta-blockers that can be used for this effect comprise non-selective agents (e.g. alprenolol, carteolol, levobunolol, mepindolol, metipranolol, nadolol, oxprenolol, penbutolol, pindolol, propranolol, sotalol, and timolol), beta-1-selective agents (e.g. acebutolol, atenolol, betaxolol, bisoprolol, esmolol, metoprolol and nebivolol), mixed alpha/beta1 blockers (e.g. carvedilol, celiprolol, and labetalol), and beta-2 selective agents (e.g. butaxamine), as well as other beta-blockers as may exist or be discovered with effects of a similar nature.

The dosage of the agent, provided generally orally in feed as a supplement, and which may also be administered via implant or suppository or intravenous injection or intramuscular injection, should be relative to the amounts normally given in human treatment for hypertension and/or anxiety control, and are preferably between 20 and 500% of normal dosage, and more preferably between 50 and 300% of normal dosage, and most preferably between 80 and 200% of normal dosage.

### Administration Period

The administration period for beta-blocker treatment can be according to two different treatment modalities. In a first treatment modality, the beta-blocker is administered for an extended period of time that can be as long as the time spent in a feedlot, which can be 3 to 4 months. In a second treatment modality, the beta-blocker is administered for a short period of time, which can be 3-4 days, so as to affect post-mortem calpastatin levels, as well as to have some short term effects on muscle hypertrophy.

In each case, the animal will generally be removed from the beta-blocker 1-3 days before slaughter, so as to allow for clearing of the agent from the animal system, such that the clearing according to the pharmacokinetics of the agent is preferably more than 75% and more preferably more than 90% and most preferably more than 95%. Indeed, the beta-blocker used can be selected on the basis of the agent's pharmacokinetics so as to effect a rapid clearing.

In the long-term treatment modality, the beta-blocker is administered for a period generally preferably of more than 1 week, more preferably more than 2 weeks, and most preferably more than 4 weeks. The conclusion of the beta-blocker administration is generally as close to the time of slaughter as possible, so that the beta-blocker effects on calpastatin levels are still in force. In practice, this depends on both the dosage of beta-blocker as well as its pharmacokinetics, but beta-blocker administration will generally cease preferably more than 1 day prior to slaughter.

As mentioned above, beta-agonists increase muscle toughness both by changing muscle fiber volume/diameter, as well as by increasing the activity of calpastatin. While short term effects of beta-blocker levels on muscle hypertrophy is small, the shorter-term effects on calpastatin levels are important. Thus, in the short-term treatment modality, animals are treated with beta-blocker for as short a time as needed to have an effect of calpastatin levels. It is preferable that for short-term administration, the duration of treatment is preferably less than 1 week, and more preferably less than 4 days, and most preferably less than 2 days. As with long-term administration, the conclusion of the beta-blocker administration is preferably as close to the time of slaughter as possible, so that the beta-blocker effects on calpastatin levels are still in force. Beta-blocker administration will generally cease preferably more than 1 day prior to slaughter.

Use in Conjunction with Beta-Agonists

The use of beta-blockers in conjunction with beta-agonists has many beneficial effects. The use of beta-agonists over an extended period of time results in a large increase in feed efficiency and muscle mass, while the use of beta-blocker over a short term can have beneficial effects in the post-mortem period. Therefore, it is a teaching of the present invention to administer beta-agonists in a conventional manner, but to replace their use with beta-blockers in the immediate pre-slaughter period as described above, so as to reduce the disadvantageous effects of the beta-agonists.

The administration of beta-blockers should be preferably started coincidently with halting the administration of beta-agonists. It is preferable that the time separating the halt of beta-agonists and the start of the beta-blockers should be between 0 days and 5 days, and more preferably between 0 days and 2 days.

### Inducing Apoptosis, a Different Method of Cell Death

When an animal dies, the tissue treats the changing environment (lower temperature, decreased aerobic environment, minimal elimination of waste) as a morbidity that must be endured. As such, cellular respiration and activity are decreased, and when cell death occurs, it occurs via necrosis—the cell dying “against its wishes”. This process occurs slowly, and involves cellular degradation that includes the calpain system that degrades the tissue slowly. Many tissues (e.g. digits and organs) can be stored at room temperature or in the cold for periods of many hours, and sometimes days or even weeks, prior to successful transplantation. It should be noted that in a muscle, for example, death occurs at different times for every muscle fiber, and it is not clear that the mechanisms are even consistent from cell to cell.

It should be understood, however, that there is an alternative method of cell death, in which signals are given to the cell to undergo apoptosis, which is a form of cell “suicide”. Unlike necrosis, which happens slowly and heterogeneously, apoptosis is programmed and is therefore the same from cell to cell, and takes place rapidly after the apoptotic decision is made. Affected cells generate a set of proteases, e.g. the caspases, which degrade cellular infrastructure and the cells break apart into vesicles amenable to phagocytosis. It should be noted that many caspases initially target cytoskeleton, which is very similar in character to that of the bulk muscle protein (i.e. actin and myosin).

### Extrinsic and Systemic Apoptosis Inducers

Apoptosis can be induced by factors both intrinsic and extrinsic to the cell. Pathways of extrinsic induction include the cytokines Tumor Necrosis Factor (TNF) for induction of TNF-induced apoptosis and FasL (FAS ligand) for induction of Fas-Fas ligand mediated apoptosis. The variant of TNF most associated with apoptosis is TNFα, and in the discussion below, the use of TNF will generally mean TNFα, although other TNF variants can, in some instances, also be of use. FasL is a transmembrane protein, and is therefore insoluble. It has a soluble counterpart produced by protease cleavage to release the external regions of the protein; the soluble FasL is somewhat less active, but has value to the extrinsic induction of apoptosis in the present invention due to its solubility. In the discussion below, FasL will be used to mean soluble FasL, but in certain circumstances, the insoluble FasL can also be used in its stead.

While TNF and FasL are the best studied and perhaps most generally active extrinsic apoptosis inducing agents, there are a host of other factors known in the art that either directly induce apoptosis, or alternatively are agonists or co-mediators of apoptosis that enhance the effects of TNF, FasL or other direct inducers. Collectively, these will be called proximate inducers of apoptosis, as the molecules and agents directly interact with the cells to produce apoptosis.

Another class of inducers are systemic inducers, and exert their effect by inducing the production of the proximate inducers through systemic effects on the organism. The systemic inducers are generally endotoxins, exotoxins, tumor products, or more generally super-antigens (immune inducers), as well as oxidizing and bleaching agents such as hydrogen peroxide, and also free fatty acids and lipid peroxides. The immune inducers include lipopolysaccharide (LPS), streptococcal superantigen (SSA), treptococcal pyrogenic exotoxins (SPEs), TSS toxin-1, and staphylococcal exotoxins (SEs). They are often associated with Toxic Shock Syndrome (TSS), Toxic Shock Like Syndrome (TSLS), and also Necrotizing Fasciitis (NF). The systemic inducers act individually or in concert to induce the production of one or more proximate inducers.

Commercial production of the proximate inducers is generally performed via bacterial clones that are engineered to produce large amounts of the inducer. Conventionally, the inducer is purified from the reactor to a purity of 95-99%, but such levels of purity result in high prices of the materials. In general, for the purposes of this invention, relatively low purity material can be used, though the material is preferably more than 5% pure, and preferably more than 10% pure, and more preferably more than 20% pure. The amount of proximate inducer that is administered is preferably more than 1× the LD₅₀ dose, and more preferably more than 3× the LD₅₀ dose, and most preferably more than 10× the LD₅₀ dose.

### Apoptosis Induction Via Immune Inducers

Production of the immune inducers is via bacterial culture, with harvesting of exotoxins from the supernatant, and purification of the endotoxins via collection of the cells followed by cell lysis either via enzymes (e.g. lysozyme) or by physical means (e.g. sonication). Methods of purification of the immune inducers is considered within the known art.

The preferred method for application of both proximate and systemic inducers is via injection into cattle prior to slaughter. This method of application allows the heart and blood system to perfuse as many organs as possible, and further allows the tissue to begin the steps of apoptosis prior to cell morbidity or death. The systemic inducers have no direct effect on muscle cells, but there must be a period during which lymphocytes and other immune cells produce the cytokines that are the proximate inducers. It should also be noted that many of these inducers, once introduced into the blood system, will not by themselves pass through blood vessel walls, but must wait for the increased leakage through blood vessel walls that accompanies the presence of the proximate inducers.

It should be noted that cytokine effects are very rapid, with death from toxic shock occurring frequently in a matter of hours from the time of the start of an acute infection, and given that the toxin concentrations are increasing rapidly during the infection, the time for acute effects may in some cases be measured in minutes.

At the time of slaughter, it is not anticipated that apoptosis will have proceeded very far. In general, it is assumed only that the proximate inducers (either injected, or produced after injection of systemic inducers) will have had an opportunity to have been transported or diffused to muscle tissue, and for some fraction of the muscle tissue to have made the cellular “decision” to engage in apoptosis. It should be appreciated that even after slaughter, while the animal may be “dead”, the tissue is still alive and metabolizing for a considerable period of time. Furthermore, the steps of apoptosis do not require large amounts of energy, and are often carried out in challenging environments (e.g. in the center of solid tumors, which have minimal blood supply and very poor oxygen levels). For example, the caspase proteases are generally already present in many cell types as precursors that are cleaved to generate the active enzymes, so that protein synthesis is not a requirement for apoptosis. Once the apoptosis decision is made, the proteolysis that proceeds can take place after slaughter.

The LD50 of LPS is on the order of 1 mg/kg. For a 500 kg animal, the LD50 would therefore be about 500 mg. If it was desired to overshoot such an amount (e.g. by 2×), the amount of inducer would need to be 1 g. This would require approximately 30 g dry weight of cells (LPS is about 3% of dry cell mass), which might cost as much as a few dollars or more, and would further cause issues in terms of the volume needed in the injection process. While techniques of genetic engineering could be used to increase the amounts of LPS, alternative methods would be useful.

This price and the difficulty of application can be considerably reduced by the use of priming. Animals that are “primed” by very small amounts of TSST-1 (on the order of 0.5 μg/kg), produce between 10 and 300 fold higher levels of proximate inducers (e.g. TNF) when subsequently challenged with LPS, or alternatively, produce the same levels of proximate inducers for much lower amounts of LPS. The priming generally takes place 2-48 hours prior to that of the secondary induction, which requires additional handling, but overall, this would allow the use of much lower levels of overall inducer use, which both lowers the cost, as well as potentially reducing any safety issues (see below).

For example, priming a 600 kg animal with 1.0 μg/kg of TSST-1 (for “strong” induction) would require 600 μg of TSST-1, which comprises about 1% of the dry cell mass of producing bacteria, and therefore would require 60 mg dry weight of bacterial cells, with negligible cost for material (though the injection may have significant operational labor costs). This can be followed then with induction with a relatively small amount of LPS. Instead of 1 mg/kg LPS required in an unprimed induction, only 10 μg/kg LPS is needed for a strongly lethal induction. This would require an equivalent cell mass of less than a gram, again at negligible cost.

It should be noted that the illustrative case of LPS in unprimed induction given above should not be considered of necessary general nature, and that each proximate and systemic inducer will have its own dosage, and some of which can be more effective than LPS. Thus, priming with TSST-1 or similar acting systemic inducer is not a necessary aspect of this technique.

It should be noted that especially for the priming dose, and intravenous application is not necessary, and subcutaneous injection, especially if given hours in advance of slaughter and the second induction, is possible. On the other hand, the priming needs to be given generally within 48 hours of slaughter. It is preferable for the inducer given just prior to slaughter to be intravenous, so that the inducer has sufficient opportunity to become generally mixed through the body. It may also be convenient to also provide the animal with a sedative at the same time, should the very rapid physiological changes that accompany the “toxic shock” cause the animal distress.

Apoptosis Induction with Oxidizing Agents

There are significant safety concerns with respect to the use of bacterial products in a commercial meat product. An alternative is to use extrinsic agents that are either non-toxic or which are not long-lasting in vivo that induce apoptosis. A number of such bleaching and mitochondriotoxic agents are know to have such effects.

The primary effect of oxidizing appears to be on oxidative damage to cells (e.g. DNA mutation or the production of lipid peroxides). An archetypal instance, of this is the effect of hydrogen peroxide, which can induce apoptosis at micromolar concentrations. This effect can also be seen, for example, with hypohalous acids (i.e. bleaching agents). In the case of hydrogen peroxide, the mechanism appears to be the generation of hydroxyl radicals via the coupled reactions of the Fenton reaction:

Fe⁺²+H₂O₂→Fe⁺³+OH⁻+.OH

Fe⁺³+H₂O₂→Fe⁺²+H⁺+.OOH

where .OH is the hydroxyl radical and where .OOH is the peroxide radical.

Inducing apoptosis with a bleaching agent generally involves administration of an amount of agent to an animal shortly before death. It should be noted that the induction can occur only in a fraction of cells, or alternatively, to a variable amount in a larger number of cells, regulated by the concentration, duration (e.g. time before slaughter), addition of agonists and antagonists of different aspects of the system, and other factors as discussed below, and yielding different palatabilities for consumers.

It is a preferable for the H₂O₂ to be administered at a concentration between 0.25 ml and 5 ml of 440 mM H₂O₂ per kg in order to induce apoptosis, and more preferably between 0.5 and 2.5 ml of 440 mM H₂O H₂O₂ per kg, and most preferably between 0.75 and 1.5 ml 440 mM H₂O₂ per kg. The concentration of the H₂O₂ can be adjusted either up or down along with corresponding inverse changes in the quantity, so as to result in the same final concentration of H₂O₂ within the animal.

Other oxidizing agents can also be used instead of hydrogen peroxide, comprising hypohalous acids and their salts (e.g. common bleach), sodium persulfate, perborate salts, and permanganate salts, as well as ozone. In the case of ozone, the reagent can either be administered in gas phase through the lungs (e.g. by placing a plastic bag over the animal's head in which air with ozone is supplied), or by taking ozone saturated solution, and administering it via catheter. The amount of agent to be used will generally be in a range determined by the 50% lethal dose (LD50), and will preferably be between 50 and 1000% of the LD50, and more preferably between 100% and 500%. It should be noted, in addition, that multiple oxidizing agents can be used in conjunction with one another. While this can be simultaneous with roughly additive effects, it can also be successive. For instance, bleach can be used as the primary agent (and may be easier to apply than peroxide, for reasons to be outlined later), but this has the disadvantage that bleach has a strong odor and has potential health concerns. Later addition of peroxide can then remove residual bleach, leaving non-toxic products.

The hydrogen peroxide and oxidizing reagents system can be affected by a number of co-factors. For example, given that hydroxyl radicals are among the more powerful and ultimate oxidizing agents, the number of hydroxyl radicals is limited by the availability of ferrous ion for participation in the Fenton reaction. It is a teaching of the present invention to provide additional free ferrous ion to the system, generally in the form of a ferrous salt (e.g. ferrous chloride). This can be accomplished before the application of hydrogen peroxide to the animal, simultaneous with the application of hydrogen peroxide (e.g. mixing the ferrous salts or a solution of ferrous salts with the hydrogen peroxide); after the application, or coincidently, such as through the use of double-lumen catheters.

Because of the release of hydroxyl ion as well as hydroxyl radical, it can be convenient to use an acid solution (e.g. acetic acid, phosphoric acid) or buffer (e.g. acetate or phosphate), in conjunction with the ferrous ion so as to limit changes in blood pH. Alternatively, it is preferable that the ferrous/ferric salt be a halide salt (e.g. ferric or ferrous chloride), or some other strong acid salt, so as to reduce the change in pH. This results in the following set of reactions (which are the first two reactions in the Haber-Weiss cycle):

Fe⁺²+H₂O₂+H⁺→Fe⁺³+H₂O+.OH

.OH+H₂O₂→H₂O+.OOH

It should be noted that instead of ferrous ion, ferric ion can also be used, as it will be converted to some extent to a ferrous state in anaerobic tissue of the animal. Thus, the amount of iron salts added with the peroxide can be less than the amount of peroxide. However, given that the last reaction in the Haber-Weiss cycle consumes the peroxide radical in conjunction with Fe+3:

.OOH+Fe⁺³→O2+Fe⁺²+H⁺

it can also be preferable for the amount of Fe+2 to be in excess over the peroxide, so that the forward reactions, resulting in peroxide and hydroxide radicals, dominate. Thus, in a cycled reaction, it is preferable for there to be an excess of peroxide over ferrous ion, wherein the molar ratio of peroxide to ferrous ion is preferably greater than 1, and more preferably greater than 5 and most preferably greater than 25. In a one-way reaction, it is preferable for there to be an excess of ferrous ion over peroxide, wherein the molar ratio of ferrous ion to peroxide is preferably greater than 1, and more preferably greater than 2 and most preferably greater than 5.

An example of the use of H₂O₂ for the induction of apoptosis is to administer through a catheter 2-mL of 220 mM H₂O₂/kg of metabolic body weight. To include ferrous ion, an alternative example of use would be to use a double-lumen catheter, in which H₂O₂ is administered as above through one lumen, and is accompanied through the second lumen of the catheter with 2-ml of 22 mM ferrous gluconate/kg. Thus, the H₂O₂ will be in roughly 10× stoichiometric excess. Yet another example of use would be the administration of H₂O₂ and ferrous gluconate as described above, but in this case, the ferrous gluconate will be in 2× stoichiometric excess. H₂O₂ will be administered on the order of 2-mL of 22 mM H₂O₂/kg of metabolic body weight, while the ferrous gluconate will be administered at 4-ml of 22 mM ferrous gluconate/kg. Alternative salts to ferrous gluconate comprise ferrous sulfate, ferrous nitrate, and ferrous chloride.

Acid can be added to the ferrous salt to reduce pH as described above. This can be hydrochloric acid or some other acid that has a relatively soluble ferrous salt, added in less than molar stoichiometry with the lesser of the molarities of the H₂O₂ or the iron salt. It is preferable that the molarity of the acid be between 0.1× and 1× the lesser molarity, and more preferably that the molarity of the acid be between 0.25× and 0.75× the less molarity.

Peroxide induces apoptosis in cell culture cells at a concentration many-fold less than that is injected into the animals as described above. While there are many reasons for this, one reason is that peroxide is being acted on by catalase and other peroxidases (more generally, “peroxide metabolases”), releasing oxygen and water, and thus reducing the effective amount of peroxide available for creation of free radicals. This can also cause foaming in the blood, which causes infarctions that reduce the distribution of peroxide and its byproducts throughout the muscle tissue.

In order to reduce the effect of catalase, glutathione peroxidase, and other peroxide metabolases, it is a teaching of the present invention to use in conjunction with hydrogen peroxide an inhibitor of catalase and/or other peroxidases, superoxide dismutases or glutathione reductases. Peroxide metabolase inhibitors that can be used include, but are not limited to, nitrite salts, thiourea, hydroxylamine salts, pyrogallol, guaicol (2-methoxyphenol), salicylic acid, ascorbate, and certain metal salts other than iron (e.g. copper). The final concentrations of these inhibitors should be enough to inhibit catalase activity by preferably 50%, and more preferably by 75% and most preferably by 90 or more %. However, because a number of these compounds also act as free radical scavengers, it is preferable not to use an excess of these inhibitors. When using these compounds, one can use the amounts of peroxide as described above, such that the effective oxidation is increased, or alternatively, can use smaller amounts of injected peroxide for the same induction effect.

It should be noted that intracellular Ca⁺² levels are critical to the decision by a cell to undergo apoptosis, and that it has been experimentally verified that increased extracellular Ca⁺² concentrations can influence that decision positively. Indeed, Ca⁺² re-perfusion of cultured cells can by itself stimulate reactive-oxygen species production and subsequent apoptosis. Increased calcium will also have positive effects on protease activity associated with apoptosis, since a number of the important caspases are Ca⁺²-dependent, as well as being an agonist for the calpain proteases. It is thus a teaching of the present invention to inject along with the peroxide or other apoptosis inducing agent (which can be a bacterial product, such as LPS) a calcium salt to raise serum levels of Ca⁺². For example, the intravenous LD50 for anhydrous CaCl₂ is about 40 mg/kg, and it is preferable for the added Ca⁺² to be injected for a final load of CaCl₂ molar equivalent of 1-100 mg/kg, and more preferably from 5-50 mg/kg, and most preferably from 10-40 mg/kg.

The mechanisms of apoptosis induction and process are known to include in general activities that result in or result from impairment of mitochondrial function or structure. A general term for agents that have such toxic effects on mitochondria are mitochondiotoxins. Many of these agents are known to induce apoptosis. Mitochodriotoxins comprise oblimersen, antimycin A, HA141-1, gossypol, ABT-737, SAHB, GSAO, CD437, arsenic trioxide, PK11195, FGIN-1-7, RO5-4864, lonidamine, 3-bromopyruvate, Rh123, MKT-077, F16, dequalinium, bistetrahydrofuranic, acetogenins, 2-methoxyestradiol, CNGRC-GG, RGD-4C-GG, BHAP, LHRH-BH3. Many of these compounds are somewhat toxic, and are therefore improper for use in meat treatment. However, others are less toxic especially in oral administration, and can be used. It is a teaching of the present invention to use mitochondriotoxins with high apoptotic potential to induce meat tenderness, and in particular those with low oral uptake. For those that are toxic, means to reduce their toxicity are described below with respect to muscle fiber fission inducers (e.g. the incorporation of substituents that have a short half-life of hydrolysis, or which are highly reactive) can also be used.

Apoptosis Induction with Lipids

A number of fatty acids are known to induce apoptosis, including saturated as well as mono- or poly-unsaturated fatty acids. Examples of these fatty acid inducers include the isomers of stearic, linoleic, docosapentaenoic, arachidonic, palmitic, oleic, and eicosapentaenoic acids, as well as conjugated versions of some of these lipids (e.g. the conjugated linoleic acids), as well as derivatives of these fatty acids, which can include esters such as methyl, ethyl, propyl, butyl esters, and amides. These fatty acids appear to be direct modulators (“free fatty acids”—FFA), and which can in cases also involve superoxide production leading to DNA and other damage, and may also include the production of lipid peroxides.

Certain non-fatty acid lipids appear to be very active in inducing apoptosis. Among these are ceramides and sphingolipids, which can comprise both natural and synthetic versions.

In general, these compounds will be injected into animals at or around the time of slaughter, at the highest concentration available, with final concentrations equal to a fraction of the LD50 for the particular agent, which is preferably between 50 and 1000% of the LD50, and more preferably between 100 and 500% of the LD50, and most preferably between 150 and 300% of the LD50. A typical LD50 is on the order of 20 mg/kg, so that an LD50 might be in the range of 4-10 gm of fatty acid for a mature cow.

A significant hurdle in injecting fatty acids is their low solubility in water. This can be approached by a variety of different means.

### Use More Soluble Fatty Acids, Ceramides and Sphingolipids

The solubility of fatty acids in water is highly affected by the number of carbons. In water, the solubility of 16-carbon fatty acids is 10 mg/L, but the solubility of 6-carbon fatty acids is about 10 g/L. Thus, the use of 4 to 6 carbon fatty acids in aqueous solution can be used.

This can also be implemented with ceramides and sphinogolipids. The natural ceramids and sphinolipids have limited solubility, but by reducing the carbon chain size, such as to three carbons (ceramide C3), not only is solubility enhanced, but also uptake by cells and the effectiveness at inducing apoptosis.

### Use of Organic Solvents

Fatty acids are generally much more soluble in organic solvents, which are conveniently ethanol or normal- or iso-butanol so as not to engender toxicity or mutagenicity issues. With these solvents, sufficient amounts of fatty acids can be provided using only 10-100 ml of solvent. However, because mixing in the blood will very rapidly dilute the fatty acid into an aqueous medium, resulting in precipitation of the fatty acids from the blood, it is important that the fatty acid/solvent mixture be injected preferably over a period of 30 seconds to 10 minutes, -and more preferably from 1 minute to 5 minutes, and most preferably over 2 minutes.

An example of such a mixture would be stearic acid in 100% ethanol, which is soluble at more than 20 g/L. Administration of 6.3 ml/kg of a 2% solution in 100% ethanol is sufficient to administer a dose that is approximately 5× the LD₅₀ of stearic acid.

### Use of Emulsions

It is also convenient to form emulsions of fatty acids in water, in which the emulsion is stabilized with emulsifiers/surfactants such as lecithin, ionic detergents (e.g. anionic detergents such as sodium dodecyl sulfate, cationic detergents such as trimethyhexadecylammonium chloride or zwitterionic detergents such as CHAPS), non-ionic detergents (e.g. pentaerithrityl palmitate or Triton X-100) or bile salts, singly or in combination with one another. Emulsions will tend to be more miscible with aqueous solutions, so the need to inject the emulsion over a period of time is less urgent. Examples of such methods include U.S. Pat. No. 6,451,339 to Patel and Chen, U.S. Pat. No. 4,572,915 to Crooks, and U.S. Pat. No. 5,364,632 to Benita and Levy.

It should be appreciated that the use of emulsions can be coupled with the use of organic solvents, such as the preparation of emulsions in ethanol/water mixtures.

Use in Combination with Peroxides

As mentioned before, lipid peroxides act directly in inducing apoptosis. Lipid peroxides are produced by the reaction of lipids with free radicals and oxygen. If free radical reagents and oxygen are present in conjunction with free fatty acids, it is possible to induce apoptosis with a smaller concentration of fatty acids. Free radicals can be generated by the Fenton reaction, which requires exogenous peroxide. Therefore, the use of hydrogen peroxide with fatty acids induces apoptosis at lower concentrations of peroxide and fatty acid than either used solely.

In general, it is not as effective to add hydrogen peroxide directly to a concentrated solution of fatty acids, as any lipid radicals that are formed by the reaction of radicals with lipids will tend to react with other radicals, quenching the radical before the reaction with oxygen to form a peroxide. Instead, it is generally preferable to add either hydrogen peroxide and then fatty acids, fatty acids and then peroxide, or to add them simultaneously, as with the use of a double-lumen catheter. As with peroxides alone, the additional injection of iron salts has the effect of improving the effectiveness of the Fenton reaction.

An example of such combination is the simultaneous administration of peroxide and stearic acid. To apply this, H₂O₂ is administered in one lumen of a double-lumen catheter, and a stearic acid will be administered in the other lumen as 6.3 ml/kg of a 1% solution of stearic acid in 100% ethanol.

In order to improve the peroxidation reaction, ferrous ion can also be administered at the same time. In this case, given a double-lumen catheter, the ferrous ion is added with the fatty acid, since its inclusion with the peroxide would degrade the peroxide (indeed, it is preferable that the peroxide solution contain a small amount of chelating agent to reduce the poisoning of peroxide with iron and other metal ions). To apply this, H₂O₂ will be administered as above in one lumen of a double-lumen catheter, and a stearic acid and ferrous gluconate will be administered in the other lumen as 6.3 ml/kg of a 1% solution of stearic acid and 5 mM ferrous gluconate in 1:1 ethanol/water mixture. The 1:1 ethanol/water mixture is used to allow solubility of both the hydrophobic fatty acid and the hydrophilic ferrous salt.

As before, the treatment mixtures can additionally include peroxidase/catalase inhibitors, acids to lower pH, and other additives discussed above.

### General Parameters for Induction

It should be appreciated that the number of potential inducers and co-factors of inducers (e.g. iron salts) is very large, and that such inducers can be used in combination as well as singly (such as the use of fatty acids and peroxide and iron salts as described in the previous section). Another example is that in the oxidation of dyes, synergy between Fenton's reagent and ozone has been noted, so that coincidental administration of hydrogen peroxide, ferrous salts, and ozone (which can be administered in gas phase) can be of benefit. Furthermore, the order of inducer injection can be varied, as well as the timing relative to the administration of each individual inducer as well as the timing relative to the death of the animal. Thus, it is impossible to enumerate all of the possible combinations of inducers that will give rise to apoptosis.

In general, however, it is possible to say that it is preferable for conditions to be used such that 25% of the muscle cells exhibit induction of apoptosis, and more preferably such that 50% of muscle cells exhibit apoptosis, and most preferably such that 80% of muscle cells exhibit apoptosis.

It should also be noted that due to the benefits and requirements of humane treatment of animals, in most cases, the apoptosis inducer will be administered after the animal has been stunned. After that point, there is usually only a limited amount of time that is either practically available on the kill floor, or before the heart goes into arrest. For that reason, the most appropriate time to administer these reagents is generally directly after slaughter, so as to provide the greatest time available for the broadest distribution of the treatment agents using the animal's heart and circulatory system.

Use of Apoptotic Agents in Conjunction with High Muscle Mass Animals

It has been noted that the application of beta-adrenergic agonists results in better feed efficiency, and more muscle mass per animal. Similar effects are shown with certain genetic strains, such as strains with variants of the myostatin gene. Together, these will be called “high muscle mass” animals. In these high muscle mass animals, the meat is much tougher, in part because of larger muscle fiber diameter, and in part due to lower calpain activity post-mortem.

It is a teaching of the present invention to use apoptotic agents in conjunction with high muscle mass animals, either agent or genetically induced. Indeed, in cases where apoptosis conditions result in too high a digestion of muscle tissue for a “normal”, untreated animal, the degree of digestion can be suitable for a high muscle mass animal. Looked at another way, if a normal animal exhibits too high a digestion of muscle tissue with the application of an apoptotic agent, instead of finding conditions that reduce the induction of apoptosis, the use of feed efficiency enhancing agents or conditions or genetic strains to increase the overall toughness of the meat might be alternatively used. To the extent that this agent, condition or genetic strain also results in better feed efficiency or muscle mass, the greater the overall benefits.

Specifically, it is considered to be of overall benefit to use beta-agonists during finishing of cattle to increase feed efficiency, to be followed by treatments to induce apoptosis, whereby the disadvantageous meat toughening effects of the beta-agonists are overcome.

### Modulating Apoptotic Effects

As mentioned above, it can be preferable to reduce apoptotic effects in case muscle tissue is “over-digested”. In such cases, it is preferable to find conditions or agents such that the apoptosis is not as extreme. It should be noted that there are two ways of affecting the degree of apoptosis: (1) through the number of cells that are affected, and (2) through the degree of effect in an average or median cell. There are a number of effects of oxidizing reagents, free fatty acids, bacterial products and the like, and such effects are not the equal for both of the two processes. Furthermore, there are other effectors that have different effects on the two processes, such as those that effect the concentration of extracellular and intracellular Ca⁺². These calcium concentration effectors can include calcium salts, chelating agents such as EDTA (e.g. injected preferably at 1-100 mg/kg and more preferably at 5-40 mg/kg), and agonists and antagonists of Ca⁺² pumps (either cell membrane, or sarcoplamic reticulum calcium pumps).

It is a teaching of the present invention to choose combinations of inducers and effectors (e.g. peroxide, iron salts, catalase inhibitors, calcium salts) that give allow for roughly independent control of the number of cells and the degree of effect. One of the most common is the use of TUNEL (Terminal deoxynucleotidyl Transferase Biotin-dUTP Nick End Labeling), which can be used in tissue cross-sections. This can be used to detect the degree of DNA damage in individual cells, thus permitting the independent evaluation of the number of cells affected, and the degree of cell apoptotic effect. In general, different concentrations of inducers and effectors are tried, with meat samples not only tested by standard means (e.g. Slice Shear Force or Warner-Bratzler test), but also preferably by taste test as well. This allows the calibration of palatability with the number of affected cells and degree of effect individually, and which can then later be used to engineer meat of highest average palatability.

### Inducing Muscle Fiber Fission

Fully differentiated myoblasts fuse to form multinucleate muscle fibers. The large diameter of these fibers contribute to the toughness of meat.

A number of agents that act on the cytoskeleton have been found to cause the fission of muscle fibers, with some inducing cell death, whereas others induce dedifferentiation an renewed cell division. Muscle fiber fission induces a reduction in tenderness. These agents are frequently microtubule interfering or depolymerizing agents. For example, treatment of muscle fibers with non-reversible depolymers vinblastine, nocodazole, colchicine, or taxol causes the disassembly of myotubes. The resulting cell masses, however, do not show muscle growth. On the other hand, treatment of myotubes with reversible depolymerizers nocodazole and myoseverin result in cell fission with cells that exhibit DNA sysnthesis and cell growth. Both classes of agent can be used for increasing meat tenderness, but because of their lethal effects, only the reversible depolymerizers can be used more than 24 hours prior to slaughter.

In general, while these agents show minimal mutagenic potential, they are to some extent toxic, which would limit their application for food animal treatment. There are two ways, however, in which the toxicity can be approached so as to render the agents suitable for treatment of meat animals.

### Differential Intravenous and Oral Toxicity

A number of the agents show differing intravenous and oral toxicity. For example, while taxol has an LD₅₀ of 33 mg/kg when intravenously administered it has little oral toxicity. An animal that is injected with taxol will therefore yield meat that is non-toxic for oral ingestion.

Another example of this is vinblastine, which has an oral LD₅₀ of 300-400 mg/kg, while the intravenous LD₅₀ is only 17 mg/kg.

It should be noted that a person is generally unlikely to consume more than 1% of their body weight in meat within a day or so (and the clearing time for most of these agents is substantially less than a day), so that an intravenous dose of 30 mg/kg given to a cow, for example, would translate at best to a 0.3 mg/kg dose to a human consumer. This is about 1000-fold below the LD₅₀, and is not known to have any effect for human consumption.

This differential between intravenous and oral toxicity is of even higher value for reversible agents such as myoseverin, since the toxicity is much less likely to have any durable or cumulative effects.

It should be noted that fission effectors that do exhibit oral uptake can be converted to ones that show little oral uptake by substitutions that (1) decrease its lipophilicity, (2) increase its hydrogen bond donor capacity, and/or (3) increase its molecular weight. There is a well-developed art for the conversion of agents to have higher oral uptake, and the same art can in this instance be used in an inverse fashion to lower the oral uptake of fission effectors. For example, with myoseverin, the N9 position can be substituted with a variety of moderate MW (>200 g/mole), hydrogen bond donating, and hydrophilic substituents.

For example, the substituent at the N9 position can comprise a base structure of linear, ringed or branched alkanes, carbohydrates, polyoxyethylene, or polypeptides. This base structure can be modified with modifiers comprising acid groups, such as phosphates, nitrates, or carboxylic acids, or which can also constitute amines, amides, alkoxy amides, or hydroxyls. It should be noted that the universe of possible base structures and modifiers is extremely large, and the examples above are only a small part of the entire range of possibilities.

It is preferable for the ratio of intravenous to oral LD₅₀ for the agent to be greater than 25, and more preferable for the ratio to be greater than 100 and most preferable for the ratio to be greater than 400.

### Hydrolyzing Substituents

Another means of making the agents safe for human consumption is to use derivatives of agents that degrade over a period of time. Let us take in theory an agent that has a half-life of 12 hours. After a period of 5 days, the effective concentration will be one-thousandth that of the original concentration, and after 10 days, will have a concentration that is one-millionth that of the original. It should be appreciated that the supply chain for most beef, e.g., is currently 14 or more days to allow for aging of the meat, and so this described time over which hydrolysis can occur is a reasonable expectation.

The formation of such modified agents is well known in the art of prodrug formulation. Prodrugs are inactive forms of drugs that are transformed in vivo into active forms of the drug. In general, the transformation is carried out through chemical or enzymatic hydrolysis of an ester, amide, or phosphate, or through enzymatic hydrolysis of a biomolecule, such as an oligopeptide.

It should be noted that in the present invention, the inverse goal of prodrugs is operative. That is, instead of injecting an inactive prodrug with the goal of having it activated in vivo, the present invention teaches the injection of an active agent, with the goal of rapid inactivation.

Lengthy descriptions of the structure of prodrugs is given in patent application US20070265295 of Kesteleyn et al, U.S. Pat. No. 5,112,739 to Meneghini and Palumbo, U.S. Pat. No. 6,624,142 to Greenwald and Zhao, US 20060234983 of Singh et al, U.S. Pat. No. 7,273,845 to Zhao and Greenwald, U.S. Pat. No. 7,262,164 to Choe and Greenwald, U.S. Pat. No. 7,087,229 to Zhao and Greenwald, and US20050182101 of Garst et al.

For example, it is a teaching of the present invention that the N2 and/or N6 moieties of myoseverin be made into leaving groups, such that on leaving, the myoseverin would lose its activity. Alternatively, the acetamide moiety of colchicine can also be made into a leaving group, and thereby reduce the activity of colchicine on hydrolysis. Similarly, there are a number of taxol moieties that would serve as suitable sites for substitution with leaving groups.

### Highly Reactive Substituents

As an alternative, the agents can be made so that they react with cellular constituents, and therefore are no longer free and toxic, thus having an effect that is similar to that of inactivation by hydrolysis. An example of this is for myoseverin, where the substituent on N9 is not strongly determinative of activity. The moiety on N9 can be made to be a thiol, a peroxide, a nucleophilic or electrophilic transfer group, azide, alkyne, carbodiimde, thiocyanate, nitro, epoxide, isothiocyanate, mesylate, tosyl, or other reactive groups or leaving groups.

In such cases, the agents can generally be stored in solution (aqueous or organic solvent) that stabilizes the compound prior to injection within the animal. For example, the solution can be at a pH that stabilizes the agent, wherein the pH is chosen with respect to the substituent used to increase hydrolysis or reactivity of the agent. Alternatively, the agent can be stored in powder form, and is then mixed with a suitable solubilizing agent (water, phosphate-buffered saline, ethanol, or other solvent) just prior to being injected into the animal.

It is convenient that the active agent have a half-life, either due to hydrolysis of the agent or due to high reactivity of the agent in vivo, before being converted to inactive form of between 10 minutes and 24 hours, and more preferably between 30 minutes and 12 hours, and most preferably between 1 hour and 6 hours.

### Agent Administration

The administration of these agents will generally be prior to slaughter, so as to allow even distribution of the agents through the animal. It should be noted that the dedifferentiation compound will generally be provided in lethal concentrations to the animal, but that the time over which the agents have their effect will vary in time. It is preferable for the animal to be alive, subsequent to administration, for the longest period possible prior to slaughter, so that the physiological effects—and primarily, the dedifferentiation and muscle fiber fission—have time to develop. Thus, it is most preferable for the agent to be administered at the earliest available time for which the overwhelming majority of animals will still be ambulatory at slaughter. This time will vary significantly depending on the agent, the dose, and the manner of administration (e.g. food versus intravenous versus intramuscular administration).

In general, the most convenient time to administer the agents is just prior to slaughter, when the animals have been collected form the pen.

The amount of agent to administer and the timing of agent administration is determined by the fraction of muscle cells that are made to undergo dedifferentiation and/or muscle fiber fission. It is preferable for this fraction to be more than 10%, and more preferable for this fraction to be more than 30%, and most preferable for this fraction to be more than 50%.

Use of Dedifferentiation Agents in Conjunction with High Muscle Mass Animals

As with the administration of apoptotic agents and beta-blockers, the administration of dedifferentiation agents in conjunction with agent treatment or genetic practices that result in high muscle mass and/or high feed efficiency animals has significant benefit. In this case, the cost advantages of high muscle mass/high feed efficiency animals are obtained, while at the same time, the use of dedifferentiation and muscle fiber fission agents results in more tender meat, thus ameliorating or reversing the disadvantageous side effects of the high muscle mass/high feed efficiency treatments.

### Summary of Terms

This Summary of Terms provides a convenient condensation of terminology used in this specification, which should not be limiting and should be considered in combination with further explication elsewhere in this specification, or as used or understood by those skilled in the art.

Normal human dosage means the dosage that is normally prescribed in humans for disease conditions (and which necessarily are far below LD₅₀ levels). For example, in the case of beta-blockers, this would be for the use of the beta-blockers in cardiac arrhythmias and for hypertension. If there is a range of accepted dosages, or treatments for a number of different disease conditions, for the purposes of this invention, normal human dosage will be the highest of these accepted dosages.

Beta-blockers comprise beta-adrenergic antagonists, which act on beta-adrenergic receptors. These generally comprise blockers that are non-selective among the beta-adrenergic receptors (i.e. beta-1, beta-2 and beta-3), as well as those that are specific for individual receptors. The beta-blockers comprise alprenolol, carteolol, levobunolol, mepindolol, metipranolol, nadolol, oxprenolol, penbutolol, pindolol, propranolol, sotalol, and timolol, acebutolol, atenolol, betaxolol, bisoprolol, esmolol, metoprolol and nebivolol), carvedilol, celiprolol, labetalol butaxamine. Beta-blockers can also have some activity against alpha-adrenergic receptors. It should be noted that beta-blockers are still in active development, and new compounds that have similar effects are also considered beta-blockers.

Beta-agonists comprise beta2-adrenergic agonists, which act on beta-adrenergic receptors. These compounds include both short-acting and long-acting beta-agonists, and comprise salbutamol, levosalbutamol, terbutaline, pirbuterol, procaterol, metaproterenol, fenoterol, bitolterol mesylate, salmeterol, formoterol, bambuterol, clenbuterol, indacaterol, ractopamine and zilpaterol.

Clearing is the removal of an agent from an animal system, through metabolism and transformation of the agent, through cellular adsorption, through excretion of the agent, or a combination of both mechanisms. Clearing is analyszed through pharmacokinetics studies. Generally, the amount of clearing is determined as a percentage fraction of the maximal blood concentration and/or activity. For instance, when the maximal blood concentration/activity has fallen by 50%, the agent can be said to have cleared 50%. It should be noted that many agents are converted into a form of similar, or even greater, activity. Therefore, the reduction is measured to the greater of activity or concentration.

Treatment refers to the administration of an agent in order to increase subsequent meat tenderness in the meat of the treated animal.

Meat tenderness refers to the tenderness of the meat at or around the time of consumption. That is, most meat is consumed after a period of “aging”, which usually lasts from 14 to 28 days. It is at the conclusion of aging that meat tenderness is of interest to the consumer or producer. Meat tenderness is usually related to an objective tenderness measurement, such as the Warner-Bratzler shear test, or the Slice Shear Force test. It is also often measured by consumer or expert taste panels.

Apoptosis inducer is a compound that, given to an animal pre-mortem, induces apoptotic cell death in the animal post-mortem. This induction can occur in two ways. In a first way, it increases the ratio of cells that die from apoptosis as opposed to necrosis (the two primary means of cell death). In a second way, it accelerates post-mortem cell death. It should be noted that the differences between necrosis and apoptosis are not absolute, and that there is a continuum between these two means of cell death, and so any treatment that accelerates post-mortem cell death through a means that is widely-held to increase apoptosis (e.g. oxidizing agents, free fatty acids, lipid peroxides, mitochondriotoxic agents) will also be considered in this context to be inducing apoptosis.

Extrinsic inducers comprise agents that are extrinsic to a cell, but that interact directly with the cell to induce apoptosis. Such agents comprise agents that interact with the cell death receptor, and comprise TNFα, FAS ligand, and the soluble FAS ligand.

Immune inducers are agents that induce apoptosis indirectly through interaction with the immune system. Such agents comprise lipopolysaccharide (LPS) and Toxic Shock Syndrome Toxin-1 (TSST-1), as well as combinations of immune inducers, such as the priming relationship between LPS and TSST-1.

Emulsifiers comprise compounds that stabilize emulsions, and comprise both synthetic ionic (e.g. sodium dodecyl sulfate), synthetic non-ionic (e.g. alkyl poly-ethylene oxide, cetyl alcohol, polysorbates, cocamide mono/diethanolamine,) and natural surfactants (e.g. fatty acid salts, lecithin).

Oxidizing agents are used in this context as agents that transfer oxygen atoms, or in a broader sense, cause the compound being acted on by the oxidizing agent to have a higher oxidation state. Oxidizing agents comprise hypochlorite, hydrogen peroxide, calcium peroxide, carbamide peroxide peroxide, and ozone. In addition, combinations of peroxides and ferrous or ferric salts (see Fenton's reagent below) also comprise oxidizing agents.

Fenton's reagent comprises a mixture of a ferrous or ferric salt, along with hydrogen peroxide. The ferrous or ferric salts induce the production of hydroxyl, and in particular, peroxide radicals.

Peroxide metabolases include any enzyme known to catalyze the decomposition of peroxides or ozones, and which generally produce oxygen and possibly water. Examples of peroxide metabolases comprise peroxidases (e.g. cytochrome C peroxidease and glutathione peroxidase), catalase, and peroxiredoxins. Peroxide metabolase inhibitors comprise nitrite salts, thiourea, hydroxylamine salts, pyrogallol, guaicol (2-methoxyphenol), salicylic acid, ascorbate, and certain metal salts other than iron (e.g. copper).

Molecular family refers to a family of compounds that are related to each other as being direct modifications of a parental molecule. For example, the molecular family of myoseverin comprises those compounds that share the myoseverin core purine structure with modifications at N2, N6 and N9. Note that the molecular family comprises compounds that do not have the same substitutions at N2, N6 and N9 as myoseverin, but which can have substitutions that have similar chemical consequences and which retain similar biochemical and/or physiological properties.

Activity half-life in vivo is a measure of how long the activity of an agent persists in vivo. This activity half-life in the context of the present invention can be related not only to the normal pharmacodynamics of the agent, but also to modifications made to an agent that relate to its hydrolysis, or which relate to its reactivity with cellular components and which thereby eliminate or reduce its activity. For example, if an agent is made with a component such that 50% hydrolyzes in vivo in 5 hours into a form with minimal activity, the activity half-life would be 5 hours. Similarly, if an agent has a modification that causes it to react with cellular constituents, thereby reducing its activity by 75% in 20 hours, it's activity half-life is therefore 10 hours.

Lipids comprise fat-soluble compounds comprising fats, oils, waxes, cholesterol, sterols, fat-soluble vitamins, monoglycerides, diglycerides, phospholipids, free fatty acids, ceramides, sphingolipids, and others. In the context of the present invention, if appears that many compounds that preferentially inserts into lipid bilayers, and in particular the mitochondrial outer membrane, are prone to induce apoptosis, and especially if these compounds are peroxidated.

Ceramide and sphingosine refer, unless otherwise noted, to the family of ceramides and sphinogsines, which can have different chain sizes and substitutents.

Muscle fiber fission inducers comprise compounds that results in the fission of massive multinucleate muscle fibers. These inducers comprise microtule polymerization inhibitors, which can comprise irreversible inhibitors such as colchicine and vinblastine, as well as reversible inhibitors, such as myoseverin and nocodazonle. Because single-nucleate precursors to muscle fibers are myoblasts, which are relatively undifferentiated muscle cells, muscle fiber fission inducers are also known as dedifferentiators.

### Many Embodiments Within the Spirit of the Present Invention

It should be apparent to one skilled in the art that the above-mentioned embodiments are merely illustrations of a few of the many possible specific embodiments of the present invention. It should also be appreciated that the methods of the present invention provide a nearly uncountable number of arrangements.

Numerous and varied other arrangements can be readily devised by those skilled in the art without departing from the spirit and scope of the invention. Moreover, all statements herein reciting principles, aspects and embodiments of the present invention, as well as specific examples thereof, are intended to encompass both structural and functional equivalents thereof. Additionally, it is intended that such equivalents include both currently known equivalents as well as equivalents developed in the future, i.e. any elements developed that perform the same function, regardless of structure.

In the specification hereof any element expressed as a means for performing a specified function is intended to encompass any way of performing that function. The invention as defined by such specification resides in the fact that the functionalities provided by the various recited means are combined and brought together in the manner which the specification calls for. Applicant thus regards any means which can provide those functionalities as equivalent as those shown herein.

What is claimed is:

1. A method for inducing tenderness in meat derived from an animal by treatment of the animal prior to its death, comprising:
administering a beta-blocker to the animal;
providing a predetermined time without beta-blocker administration to allow for clearing of the beta-blocker from the animal; and
slaughtering the animal.

2. The method of claim 1, wherein the predetermined time is less than 4 days.

3.-4. (canceled)

5. The method of claim 1, wherein administering is selected from the group consisting of oral administration, implantation, placement as a suppository, intravenous injection and intramuscular injection.

6. The method of claim 1, additionally comprising providing beta-agonist to the animals, wherein the provision of beta-agonist is prior to the administration of the beta-blocker.

7. A method for inducing tenderness in meat derived from an animal by treatment of the animal prior to its death, comprising:
administering to the animal an apoptosis inducer; and
slaughtering the animal.

8. The method of claim 7, wherein the apoptosis inducer is selected from the group consisting of an extrinsic inducer and an immune inducer.

9. (canceled)

10. The method of claim 8, wherein the inducer comprises an oxidizing agent.

11. The method of claim 10, wherein the oxidizing agent is selected from the group consisting of hydrogen peroxide and ozone.

12. The method of claim 11, wherein the apoptosis inducer further comprises a peroxide metabolase inhibitor.

13. The method of claim 8, wherein the apoptosis inducer comprises a lipid.

14. The method of claim 13, wherein the lipid is selected from the group consisting of free fatty acid, ceramide, and sphingosine.

15. The method of claim 13, wherein the lipid is solubilized in aqueous solution with an emulsifier.

16. The method of claim 13, wherein the lipid is administered as a solution in a solvent selected from the group consisting of ethanol, n-butanol, and iso-butanol.

17. The method of claim 8, wherein the apoptosis inducer comprises a peroxide and a salt selected from the group consisting of ferrous salt and ferric salt.

18-19. (canceled)

20. The method of claim 8, additionally comprising providing beta-agonist to the animals, wherein the provision of beta-agonist is prior to administering the beta-blocker.

21-26. (canceled)

27. A compound for administration to a live animal for increasing post-mortem meat tenderness, comprising an apoptosis inducer.

28. The compound of claim 27, wherein the apoptosis inducer comprises an oxidizing agent.

29. The compound of claim 28, further comprising a peroxide metabolase inhibitor.

30. The compound of claim 28, where the oxidizing agent is selected from the group consisting of hydrogen peroxide, ozone, and Fenton's reagent.

31. The compound of claim 27, wherein the apoptosis inducer comprises a lipid.

32. The compound of claim 31, where the lipid is selected from the group consisting of free fatty acid, ceramide and sphingosine.

33. The compound of claim 27, wherein the apoptosis inducer comprises an oxidizing agent and a lipid.

34.-41. (canceled)
